(19) United States
(12) Patent Application Publication
(10) Pub. No.: US 2009/0124513 A1
(43) Pub. Date: May 14, 2009

(54) Multiplex Biosensor

(76) Inventors: Patricia Berg, Accokeek, MD (US); Mona Zaghloul, Bethesda, MD (US); Onur Tigli, Arlington, VA (US)

Correspondence Address: JUNEAU PARTNERS, P.O. BOX 2516, ALEXANDRIA, VA, 22301, US

(21) Appl. No.: 12/166,601

(22) Filed: Jul. 2, 2008

Related U.S. Application Data
(63) Continuation-in-part of application No. 11/738,460, filed on Apr. 20, 2007.

(51) Int. Cl. C40B 30/04; B01J 19/00; C40B 60/12; G01N 33/543

(52) U.S. Cl. 506/9; 422/68.1; 436/518; 506/39

(57) ABSTRACT

This invention relates to CMOS SAW-based biosensor devices for detecting analytes and biomolecules of interest.

### CROSS REFERENCE TO RELATED APPLICATIONS

This application is a continuation-in-part application and claims priority benefit of the earlier filing date under 35 USC 120 of U.S. patent application Ser. No. 11/738,460 filed 20 Apr. 2007, the content of which is incorporated herein in its entirety.

### STATEMENT REGARDING FEDERALLY SPONSORED RESEARCH OR DEVELOPMENT

No federal government funds were used in researching or developing this invention.

### BACKGROUND

1. Field of the Invention

This invention relates to a multiplex biosensor, namely, a CMOS integrated circuit having a SAW device as an on-chip component capable of direct electronic readout and configured for parallel multi-analyte, multi-concentration detection assays, methods of providing sensitive and specific assays to detect biomolecules, including serum tumor markers that could be used in early detection of cancer.

2. Background of the Invention

The current state of knowledge is as follows.

### Serum Biomarkers

Serum markers are used in the diagnosis and monitoring of metastatic disease. It is clear that increasing the sensitivity of assays for serum biomarkers would be advantageous and allow their use in early detection as well. (i) Early detection. The most widely used serum tumor markers in breast cancer are CEA (an oncofetal protein), CA15-3, CA27.29 (both markers for mucin), TPA, TPS and HER-2 (shed form). However, it is widely agreed that all of these markers currently lack the sensitivity for early detection. Not only are additional biomarkers needed, but also sensitive assays, such as the biosensor provided here. (ii) Prognosis. It is estimated that 20-30% of lymph node negative (LNN) breast cancer patients ultimately develop metastatic disease. Currently, all patients are treated since there is no simple method for detecting micrometastases. In fact, it has been suggested that more than 80% of patients in the average-risk group, classified according to the St. Gallen system, would have survived without adjuvant therapy. A sensitive, serum-based assay could be very useful in identifying patients whose tumors are likely to or have already metastasized. (iii) Monitoring therapy. In the past, both CEA and CA15-3, in combination, have been used to monitor metastatic breast cancer. Expression of serum biomarkers would be expected to decrease after successful treatment, which was indeed observed for both CEA and CA15-3. Conversely, useful biomarkers would be predicted to increase upon relapse or metastasis. In three small studies, survival of patients increased who were given salvage treatment based only on increasing concentrations of CEA, CA15-3 or TPA. However, based on these small numbers of patients, the use of tumor markers for routine surveillance or monitoring after treatment is controversial since it involves treatment of asymptomatic patients. More patients and additional markers need to be tested to resolve this issue; a high throughput assay would be extremely useful in this regard.

### Comparative Assays

Comparisons with currently available assays. Table 1 displays the main approaches currently used to identify and detect biomarkers in serum. (i) ELISAs are referred to as the ‘gold standard’ technique and are used in clinical settings. The sensitivity can be as low as 1 pg/ml or as high as 4 ng/ml, depending on the antibody (Ab). However, ELISAs require larger sample volumes than antibody arrays and are not suitable for multiplexing. (ii) Ab arrays provide fairly high sensitivity (although less than ELISAs), good reproducibility and multiplexing capabilities, but they are not “user friendly”. They require extensive sample preparation involving protein isolation, labeling of proteins using dyes followed by incubations with the array itself and then analysis in a microarray scanner with specialized software. The results require normalization and only provide a ratio relative to a normal control. The biosensor, on the other hand, would require no further sample manipulation after serum preparation; an automatic read-out would give the change in frequency resulting from binding of a serum protein. (iii) MALDI-TOF mass spectrometry can be used to correlate protein patterns in biological fluids with certain types of cancers. However, it requires extensive sample preparation, is semi-quantitative, preferentially detects lower MW proteins, and an experienced operator is needed. (iv) Surface plasmon resonance (SPR). This label-free technology has been used for a number of applications, ranging from analysis of biomolecular interactions and kinetic rate constants to detection of enterotoxins or detection of bacteria. However, its sensitivity is similar to or less than ELISAs, e.g., it was able to detect only 11 ng/ml HER2. Many investigators use Biacore (www.biacore.com), an instrument based on SPR. The BiacoreX instrument allows comparison of only two Ab, and costs $115,000; chips are additional; Biacore A100 offers up to 20 channels but costs $700,000; chips cost from $470 to $1590. Our integrated, multiplexed biosensor, including electronic read-out in a single unit, should cost considerably less and will be smaller than a microprocessor.

TABLE 1
Methods for Detection of Biomarkers in Serum

| Method of | | | |
|---|---|---|---|
| Detection | Advantages | Disadvantages | Sensitivity |
| ELISA | undetectable non- | greater sample volume | lower limit of quantitation ~1 pg/mL |
| | specific binding in | cannot be multiplexed | dynamic range of 3 logs |
| | dynamic range | | |
| | quantitative | | |
| MALDI- | good for | extensive sample preparation | less sensitive than ELISA |
| TOF MS | discovering new | semi-quantitative | |
| | biomarkers | preferential detection of | |
| | | lower MW proteins | |
| | | experienced operator needed | |
| Antibody | low sample | time-consuming sample | lower limit of detection |
| Arrays | volumes | prep. | ~20 pg/mL |
| | good | only relative antigen | |
| | reproducibility | amounts | |
| | multiplex | requires microarray scanner | |
| | capability | | |
| Surface | label free | expensive | 0.1 nM if >10 kDa |
| Plasmon | | sample volume ≧20 μl | 1.0 nM if <10 kDa |
| Resonance | | (sample and buffer) | |
| (SPR) | | | |

### Biosensors

Acoustic wave sensors use a detection arrangement that is based on perturbations to mechanical or acoustic waves. As an acoustic wave propagates through or on the surface of the acoustive wave sensor material, any changes to the physical or chemical characteristics of the wave path may affect the velocity and/or amplitude of the acoustic wave. These changes may be correlated to the corresponding physical, chemical, or biological quantities being measured to provide sensing.

There may be various biological and chemical sensors, using fiber optics, chemical interactions, and various fluorescence approaches. Such sensors may, however, have various weaknesses, such as, for example, low sensitivity, selectivity, or an inability to be hybridized or integrated into sensing chip technology.

### Acoustic Wave Sensors

Acoustic wave (AW) sensors, however, may be better suited for use in biological and chemical detection. As discussed in D. S. Ballantine, R. M. White, S. J. Martin, A. J. Ricco, E. T. Zellers, G. C. Frye, H. Wohltjen, “Acoustic Wave Sensor—Theory, Design, and Physico-Chemical Applications”, Academic Press, (1997), acoustic wave sensors may use piezoelectric crystals, which may allow transduction between electrical and acoustic energies. The AW sensor may use piezoelectric material to convert a high frequency signal into an acoustic wave, and the higher frequency may enable the sensor to be more sensitive to surface perturbations.

Piezoelectric materials used for acoustic wave sensors may include quartz (SiO₂), lithium niobate (LiNbO₃), zinc oxide (ZnO), and others. Each of these materials may possess specific advantages and disadvantages, which may relate to, for example, cost, temperature dependence, attenuation, and propagation velocity. Such materials may, however, have varying transverse acoustic wave velocities, low electromechanical coupling coefficients, non-linear temperature coefficients, and may react chemically with the environment. (See the background information in C. Caliendo, G. Saggio, P. Veradi, E. Verona, “Piezoelectric AlN Film for SAW Device Applications”, Proc. IEEE Ultrasonic Symp., 249-252, (1992) and K. Kaya, Y. Kanno, I. Takahashi, Y. Shibata, T. Hirai, “Synthesis of AlN Thin Films on Sapphire Substrates by Chemical Vapor Deposition of AlCl.sub.3 —NH.sub.3 Systems and Surface Acoustic Wave Properties”, Jpn. J. Appl. Phys. Vol. 35, 2782-2787, (1996) and G. Carlotti et al., “The Elastic Constants of Sputtered AlN Films”, Proc. IEEE Ultrasonic Symp., 353, (1992)).

### SAW Integration

Previously, creation of SAW devices has been complicated and, in the case of CMOS fabrication, it has been unworkable as the chip would be destroyed by the temperatures required to integrate the SAW device.

### BRIEF SUMMARY OF THE INVENTION

Accordingly, the present invention provides a multiplex biosensor systems employing a CMOS manufactured integrated circuit having a SAW device as an on-chip component, and when configured for parallel multi-analyte, multi-concentration detection assays, methods of providing sensitive and specific assays to detect biomolecules, including serum tumor markers that could be used in early detection of breast cancer.

The biosensor is manufactured using CMOS technology and provides, along with the ever-developing CMOS compatible MEMS processes, an advantage that the SAW technology performance is improved significantly. Therefore, an array of SAW delay lines were designed, fabricated—through a commercially available CMOS process sequence-characterized and post-processed using widely used MEMS techniques. The post processing steps are fully optimized and characterized. Electrical characterization of the devices was carried out and performance results were obtained. The results show close agreement with the preliminary modeling as well as calculated results.

In a preferred embodiment is provided a biosensor device, comprising: a CMOS integrated circuit having a SAW device as an on-chip component and configured for an analyte detection assay, wherein the SAW device has an Si substrate having a piezoelectric film thereon, said film supporting an input IDT and an output IDT in cooperative association, and having a biochemical coating there-between for detecting at least one analyte; wherein the biochemical coating acts as an analyte capture binding surface, and wherein upon binding of said analyte to said biochemical coating a surface acoustic wave signal is generated that is indicative of the presence of the analyte.

In preferred embodiments, the biosensor further comprises wherein the device is configured for parallel multi-analyte, multi-concentration detection assays.

In preferred embodiments, the biosensor further comprises wherein the configuration comprises wherein the piezoelectric film comprises a micromachined matrix of addressable microlocations for analyte capture.

In preferred embodiments, the biosensor further comprises wherein the biosensor has a plurality of detection channels, said detection channels having a plurality of biochemical coatings specific for detection of a plurality of proteins, and which provides for a multi-protein multi-concentration detection array capable of parallel simultaneous read-out.

In preferred embodiments, the biosensor further comprises wherein the biochemical coating is for detecting at least one serum tumor marker protein for cancer.

In preferred embodiments, the biosensor further comprises wherein the piezoelectric film is deposited on top of the IDT.

In preferred embodiments, the biosensor further comprises wherein the device is configured to provide an electronic readout of the concentration of serum tumor marker as determined from the strength of the SAW signal generated.

In preferred embodiments, the biosensor further comprises a method of detecting biomolecules, comprising: contacting the analyte capture binding surface of the biosensor device of claim 1 with a sample of interest.

In preferred embodiments, the biosensor further comprises wherein the sample is a serum protein cancer marker.

In preferred embodiments, the biosensor further comprises a CMOS manufactured embedded heater under the SAW sensor, wherein the heater is controlled by applying outside voltage, and wherein control of the sensor temperature to the desired value is provided to keep the sensor operating point stable over time.

In preferred embodiments, the biosensor further comprises wherein the analyte is a marker protein selected from the group consisting of mammoglobin, BP1, CEA, CA15-3, TPA, TPS, HER-2, and combinations thereof.

In preferred embodiments, the biosensor further comprises wherein the biosensor device provides an electronic readout and is implemented in a single unit or kit. In preferred embodiments, the biosensor further comprises a method of detecting a serum tumor marker for breast cancer, comprising:
a) providing a biosensor as described herein;
b) contacting the biochemical coating of the biosensor with a sample; and
b) detecting the SAW signal,
wherein upon binding of a serum tumor marker to said biochemical coating then a surface acoustic wave signal is generated that is indicative of the presence of the serum tumor marker protein, and wherein upon the absence of binding of the serum tumor marker, then no signal is generated and indicates the absence of said serum tumor marker protein.

In preferred embodiments, the biosensor further comprises a method of detecting multiple proteins in samples having multiple concentrations in a multiplex biosensor, comprising:
a) providing the multiplex biosensor as described herein;
b) contacting each of the plurality of detection channels of the biosensor with sample; and b) detecting the SAW signal,
wherein upon binding of a specific serum tumor marker to said biochemical coating then a specific surface acoustic wave signal is generated that is indicative of the presence of the specific serum tumor marker protein, and wherein upon the absence of binding of the specific serum tumor marker, then no signal is generated and indicates the absence of said specific serum tumor marker protein, and wherein the multiplex biosensor provides for a multi-protein detection array capable of parallel simultaneous read-out.

In preferred embodiments, the biosensor further comprises the step of determining the concentration of the analyte or serum tumor marker from the strength of the SAW signal generated.

In a preferred embodiment, the heat control structure built within the substrate silicon during the CMOS process and can be adopted in any of the CMOS chip devices herein. In preferred embodiments, the heat control structure is an n-well layer that has a TCR of 0.5-0.75%/K, which is the highest among various CMOS process layers and wherein the n-well provides an embedded heater structure that can directly control the temperature of the substrate and the mass sensitive area without causing any disturbance on the SAW delay line path or the IDT finger design.

Preferred uses of the devices herein include SAW based integrated circuit detection systems which comprise: a sensor having at least one sensing element for selectively combining with target molecules, said sensor generating a signal when combined with said target molecules responsive to incident electromagnetic radiation applied to said sensor/target combination; and an integrated circuit microchip to which the sensor is affixed, the integrated circuit microchip including: a plurality of detection channels operatively associated with said sensing elements, each of said detection channels including a detector for detecting electromagnetic signals, said detectors selected from the group consisting of photodiodes and phototransistors. In preferred uses the sensor is a sensor for detecting proteins associated with breast cancer such as Mammoglobin or BP1, and comprises a chemical receptor, a bioreceptor, a polymer, a biopolymer, a molecular imprint polymer, a biomimetic, an antibody, an enzyme, a cell receptor, a molecular print assay, or a nucleic acid.

For detection of the target, phase shift detection or alternatively frequency shift detection can be used within the SAW device.

The systems herein can also preferably be implemented in a hand held unit or in a kit. Thus, as oscillator or filter etc. or alternatively as a bio/chemical sensing device.

Preferrably, the integrated circuit includes wherein the breast cancer protein sensor comprises a chemical receptor, a bioreceptor, a polymer, a biopolymer, a molecular imprint polymer, a biomimetic, an antibody, an enzyme, a cell receptor, a molecular print assay, or a nucleic acid, or a thin film which comprises one of the above, or an analyte well comprising one of the above where the analyte is cooperative contact with the SAW device, e.g. IDT fingers.

In another preferred embodiment is provided a method of detecting at least one analyte of interest, comprising: a) contacting a biochemical coating with an analyte of interest, wherein the biochemical coating is on a piezoelectric film, said film supporting an input IDT and an output IDT in cooperative association with said biochemical coating, wherein the biochemical coating acts as an analyte capture binding surface for detecting the analyte of interest, and wherein upon binding of said analyte to said biochemical coating a surface acoustic wave signal is generated that is indicative of the presence of the analyte; and b) detecting the SAW signal.

### BRIEF DESCRIPTION OF THE DRAWINGS

FIG. 1 is a drawing showing the basic principle of a SAW sensor.

FIG. 2 (a) is a top view of the circular architecture embodiment of the SAW device.

FIG. 2(b) is a scanning electron micrograph image of the input/output IDTs and the pads.

FIG. 3 is a depiction of the parts of a biosensor as contemplated herein on one preferred embodiment.

FIG. 4 is a cross-sectional diagram of a completed biosensor.

FIG. 5 is a photograph showing the size of a typical CMOS-SAW chip placed on a nickel (U.S. 5 cent piece).

FIG. 6 is a graph showing the frequency change response of circular CMOS-SAW devices.

FIG. 7 is a photo of a Western Blot assay showing secretion of pBP1 by breast cancer cells and two leukemia cell lines NB4 and R4 and shows the comparison of malignant cells vs. normal epithelial cells for all 3 cancers.

### DETAILED DESCRIPTION OF THE INVENTION

It is clearly advantageous to develop a non-invasive assay which could be useful in the early detection, determining prognosis and monitoring treatment of breast cancer. We provide a bioassay using chip technology for detecting secreted proteins in breast cancer. The biosensor represents a novel device that is integrated with standard electronics measurement circuits. The principle provided is simple, label free technology, and fast, requiring only small amounts of material. It does not require color detection instrumentation or special reading equipment, a major advantage over current methods such as arrays. It is also very small and the cost to manufacture is low providing significant commercial advantage.

Chip technology also allows multiple Abs to be attached to each chip. It is well established that multiple biomarkers are advantageous, e.g., CEA is elevated in 30-50% of symptomatic metastatic breast cancers, but the sensitivity of detection can be increased to over 80% when both CEA and CA15-3 are used. A combination of markers on this biosensor, detected with specificity and sensitivity, is useful in detecting metastasis. In alternative preferred embodiments, the biosensor has two Abs, for example hMAM and BP1 hMAM is a promising new biomarker and BP1, which plays a role in breast cancer and is activated in other cancers (acute myeloid leukemia and prostate cancer (unpublished)). Either of these markers, in combination with other markers (e.g., CEA or CA15-3), provide a powerful new assay. In addition, the principles of this bioassay are applicable to other cancers through the use of markers associated with them.

### SELECTED DEFINITIONS

The following definitions are provided as an aid to understanding the detailed description of the present invention.

As used herein, the terms “analyte,” “target” and “target analyte” means any protein that is associated with breast cancer. Non-limiting examples include mammoglobin and BP1.

As used herein, a “binding moiety” is a molecule or aggregate that has binding affinity for one or more target analytes. The term “binding moiety” is synonymous with the terms “biomolecule” or “ligand” as used herein. Within the scope of the present invention virtually any molecule or aggregate that has a binding affinity for some target analyte of interest may be a “binding moiety.” In preferred embodiments, the “binding moiety” is an antibody. In other preferred embodiments, the binding moiety is specific for binding to a single target analyte, although in other embodiments the binding moiety may bind to more than one target analyte exhibiting similar structures or binding domains. With respect to antibody binding, it is anticipated that multiple analytes may exhibit similar or identical antigenic epitopes, resulting in potential cross-reactivity of the binding moiety for related analytes.

“Binding” refers to an interaction between a target analyte and a binding moiety, resulting in a sufficiently stable complex so as to permit detection of the analyte; ligand complex. In certain embodiments, binding may also refer to an interaction between a second molecule and a target analyte. For example, in a sandwich ELISA type of detection assay, the binding moiety is an antibody with affinity for an analyte. After binding of analyte to binding moiety, a second molecule, typically a labeled antibody with an affinity for a different epitope of the analyte, is added and the tertiary complex of first antibody:analyte:second labeled antibody is detected. In alternative embodiments, the first binding moiety may have affinity for a target analyte while the second binding moiety has affinity for the first binding moiety. Although detection may involve the use of a second binding moiety with affinity for an analyte, in alternative embodiments the binary complex of binding moiety with analyte may be directly detected. The skilled artisan will be familiar with a variety of techniques by which an analyte:ligand complex may be detected using SAW devices, any of which may be utilized within the scope of the present invention.

The terms “detection” and “detecting” are used herein to refer to the generation of a signal on the SAW device that is indicative of the presence of one or more specific analytes in a sample, or that predicts a disease state or a medical or environmental condition associated with the presence of one or more specific analytes in a sample. It will be appreciated by those of skill in the art that all assays exhibit a certain level of false positives and false negatives. Even where a positive result in an assay is not invariably associated with the presence of a target analyte, the result is of use as it indicates the need for more careful monitoring of an individual, a population, or an environmental site. Such an an assay is diagnostic of a disease state or a medical or environmental condition when the assay results show a statistically significant association or correlation with the ultimate manifestation of the disease or condition.

### Surface Acoustic Wave (SAW) Devices

Referring now to the figures, SAW (Surface Acoustic Wave) devices are widely used as electronic filters, delay lines, and resonators in today's communication systems. Although the telecommunication industry is the largest user of these devices, SAW based sensors have many attractive features to be explored for emerging technologies in automotive, medical (biosensor), industrial and commercial (vapor, gas, humidity) applications. They are small, inexpensive, can easily be designed for responding to various measurands, have wide dynamic range and are passive devices which can also be deployed as wireless units. They have unique resonance frequency which is determined by the dimensions of the design.

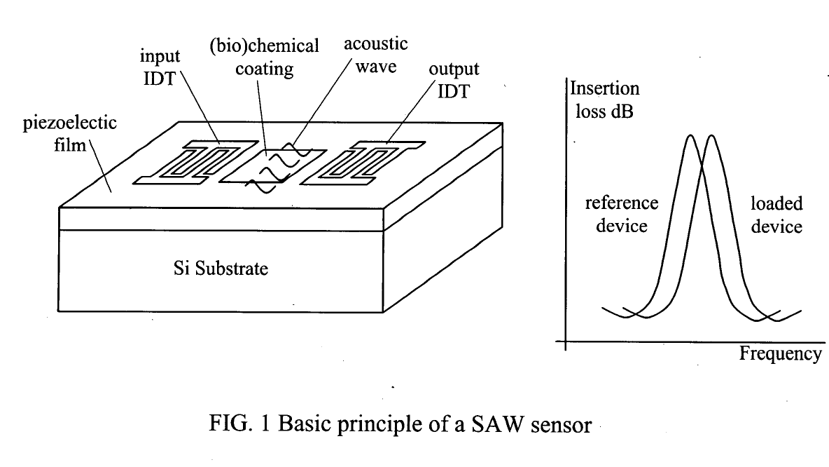

One of the most widely used and interesting sensing mechanisms that acoustic wave sensors employ is mass loading. Prominent applications are in film thickness monitoring, gas, liquid phase chemical sensing and biosensing. The delay lines of SAW devices are coated with some (bio) chemical coating which selectively reacts with the entity under analysis. This interaction produces a shift in the resonant frequency of the SAW device. By measuring this shift in frequency domain, a detailed analysis of the entity being sensed can be completed. FIG. 1 depicts the basic principle of SAW based (bio)chemical sensors employing the mass loading scheme.

Successful SAW based sensors to measure density, viscosity of liquids, determine the properties of polymer films, quantitative detection of the concentrations of volatile organic vapors, detect the output of gas chromatographic columns, and both selective and sensitive detection of biochemically active compounds have been demonstrated in the literature http://www.sandia.gov/mstc/technologies/microsensors/sh-saw.html). Most of these devices are designed using full custom fabrication methods. Moreover, developing SAW devices on Si requires several fabrication steps including deposition, etching and patterning of various materials. Therefore, designing these devices in full custom fashion requires extra effort and increases the cost of production. There is a great interest in developing methods that would comply with current mass production standards in electronics without disturbing the performance of well characterized SAW devices. The major challenge is to design a well defined and characterized fabrication process (including the post processing steps) that would allow the designers to build SAW devices with comparable performance to the currently available off the shelf products.

### Integrated Circuits

CMOS (Complementary Metal Oxide Semiconductor) technology is the most widely used technology in the current ASIC (application specific integrated circuit) industry due to its low power, high performance, and high levels of integration capabilities. Designers have access to cutting edge, commercially available, standardized CMOS technologies at very low costs. Moreover, recent developments in the area of silicon micromachining and MEMS (Micro Electro Mechanical Systems) allow high precision fabrication methods for developing intelligent sensor systems with monolithic integration capabilities.

SAW based piezoelectric transduction has various advantages over other mechanisms-electrochemical, optical, magnetic, thermal—employed in biosensor research. The micron-scale dimensions of these devices provide a larger surface area to mass ratio which implies higher sensitivity. Moreover, they provide label free, robust detection of the analyte of interest.

CMOS technology also provides cost effective mass production solutions. In addition to these attractive aspects, they are more than mere mass sensors since the sensor response is also influenced by interfacial phenomena, viscoelastic properties of the adhered biomaterial, surface charges of adsorbed molecules and surface roughness.

The sensitivity of piezoelectric transducers is directly proportional to the square of the operating frequency. CMOS-SAW devices that are designed for this work have operating frequencies in 400-500 MHz range which make them extremely attractive for highly selective biosensing applications such as the one provided in this work.

In addition the use of CMOS technology allows the use of an embedded heater under the SAW sensor. This heater is controlled by applying outside voltage, and thus it is possible to control the sensor temperature to the desired value to keep the sensor operating point stable over time.

The SAW biosensor provided herein measures proteins secreted in sera from breast cancer patients. The first protein tested was mammaglobin.

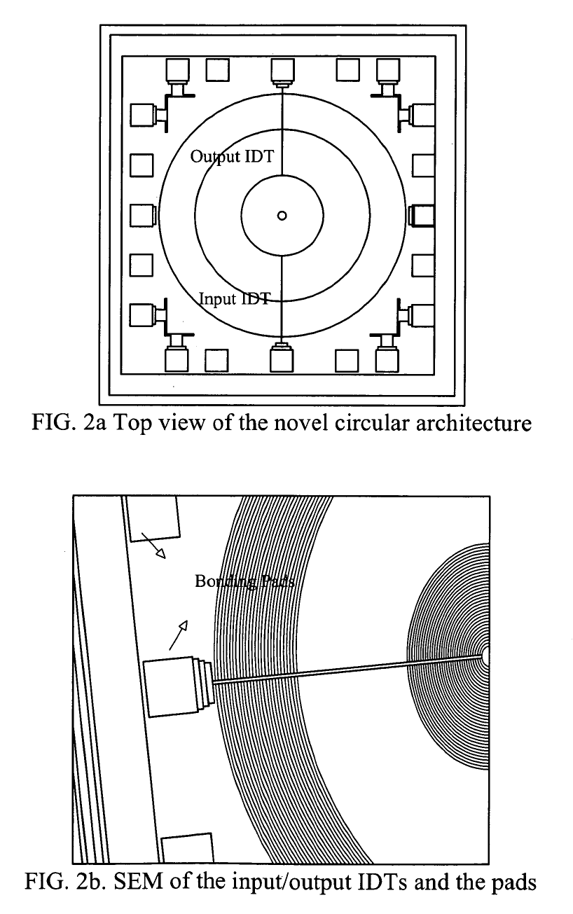

Referring to FIG. 2, we developed two micro structures of SAW biosensors, rectangular and circular SAW. The circular design is novel SAW design, and is very sensitive design. Preliminary measurements indicate higher sensitivities as compared to the rectangle structure. Therefore, in a non-limiting preferred embodiment, the SAW device may have a circular design.

FIG. 2.a depicts the snapshot of the circular architecture and FIG. 2.b presents the SEM of the Input and Output IDTs. This novel architecture consists of concentric circular IDT structures that are aligned on a common focal point. In contrast to the conventional rectangular SAW devices, the input and output IDT are not physically equal in size. The input IDT—the outer circle fingers-are much larger in size than the output IDT—the inner circle fingers. This way, a smaller amount of input power will be used to generate effectively the same acoustic wave as the conventional rectangular devices. Moreover, the reduction in the electromechanical conversion due to anisotropy is eliminated since the wave will be released from every possible angle of the crystal. The concentric nature of the novel architecture will also eliminate the wave spreading problem due to finite aperture. Instead of spreading the wave on the delay line it will concentrate the acoustic waves to a focal point. Due to the circular nature of this architecture, the effective sensing area is also increased when compared to a regular rectangular delay line with the same dimensions. In addition, this architecture provides a spatially uniform sensitive surface for detection of any analyte that is introduced on the sensor area.

### SAW Devices as Biosensors

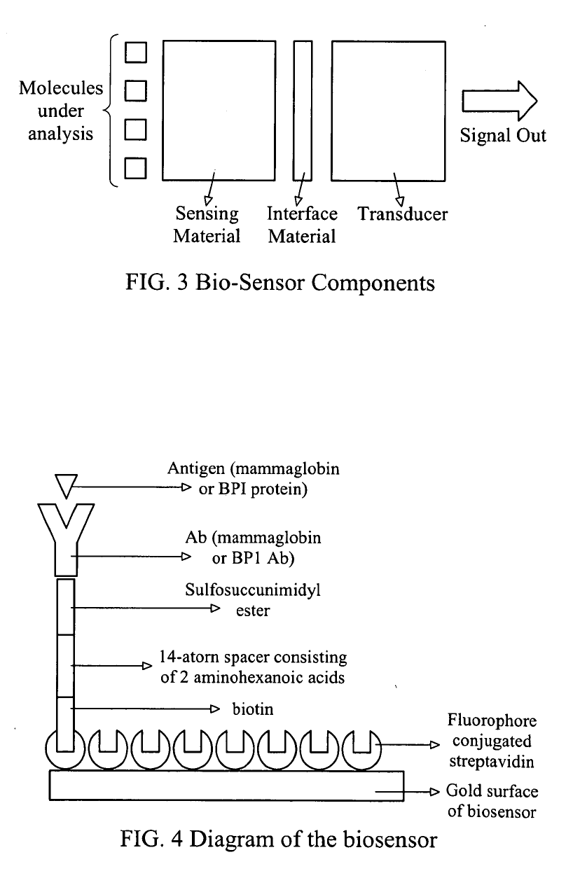

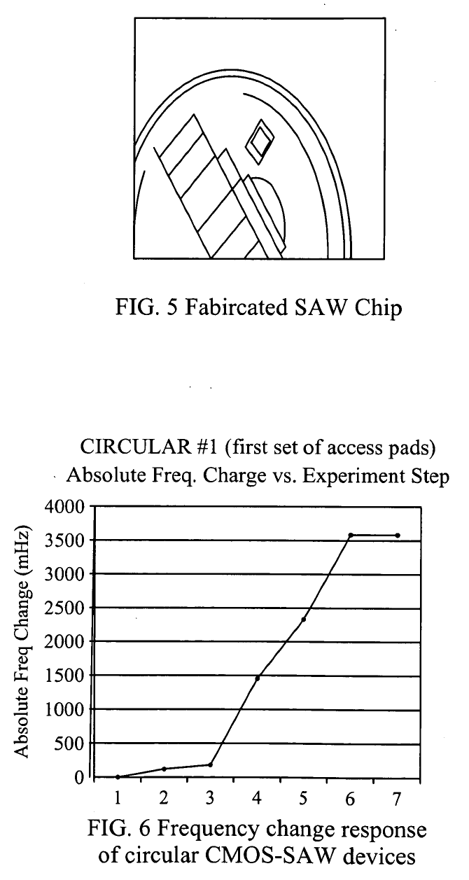

Referring now to FIG. 3, the elements of a biosensor are depicted in FIG. 3. All three components play a crucial role in the sensitivity of the entire system. The primary interests in this aim are optimization of the transduction mechanism and the adaptation of this transduction mechanism for a biosensor application.

In order to increase the sensitivity of these devices to low volume samples, several techniques are employed. (1) As the work consists of liquid phase detection, the SAW devices are designed to utilize pure shear-waves. SH-SAW (shear-horizontal), APM (acoustic plate mode) and Love wave modes are the primary options. These modes are obtained by altering design dimensions (plate thickness), introducing new layers (metal, oxide) and modifying the post processing steps. (2) Proof of concept devices may be packaged in DIP-40 ceramic packages for electrical testing, but in order to accommodate the fluidic nature of the biological testing, design options include providing a reservoir for the antibody-antigen interactions, such as a pyrex chamber that would stick to the active surface of the sensors. (3) In order to prevent any liquid sample from coming in contact with the bonding wire that connects the sensors to the package pins, a thick photoresist based isolation is employed to cover the bonding wires and provide electrical isolation. (4)

### Serum Proteins

### EXAMPLE

### Mammoglobin (hMAM)

Mammaglobin is a secreted protein associated with breast cancer and expression is specific for breast carcinoma, with infrequent expression in uterine carcinoma. In one study, immunohistochemical (IHC) staining demonstrated hMAM expression in 72% of primary breast tumors. By quantitative RT-PCR (QRT-PCR), greater than 90% of tumors were hMAM positive, and there was a correlation between hMAM expression and ER positive tumors. Examination of blood from breast cancer patients by RT-PCR showed only 8% were hMAM positive, with a correlation between increased expression and more than four positive lymph nodes at diagnosis, linking hMAM expression with metastasis. Recently, an ELISA was developed to measure hMAM in sera; the sera from all 26 women with metastatic breast cancer were hMAM positive. It was concluded that hMAM is a new serum biomarker with utility in the early detection of recurrence and as an indicator of treatment response.

### EXAMPLE

### BP1

BP1 exhibits: (a) high frequency of expression in invasive ductal breast tumors; (b) significant association with ER negative tumors, grade III tumors, lymph node positivity and race; (c) association with aggressive characteristics in MCF7 cells, including stimulation of tumor formation in the absence of exogenous estrogen in mice; (d) association with the bcl-2 anti-apoptotic pathway. Since BP1 appears to be secreted, we predict it will be detectable in sera from breast cancer patients, especially metastatic breast tumors. Depending on the sensitivity of the assay, it may be detectable in early tumorigenesis, making it a potentially useful biomarker for early detection. Serum pBP1 might also be a useful tool for monitoring therapy.

Further, BP1, is reported as a novel homeobox (HB) gene. HB genes encode transcription factors that activate or repress groups of target genes involved in differentiation and development. Interestingly, BP1 has been mapped to chromosome 17q21, near the oncogene HER-2 and the tumor suppressor gene BRCA1.

Important characteristics of BP1 include the following.

(1) BP1 is expressed in 80% of invasive ductal breast tumors.

Aberrant expression of BP1 has been shown in two populations of women with breast cancer: 46 untreated invasive ductal breast tumors were evaluated for BP1 levels using semi-quantitative RT-PCR, and 100 invasive ductal carcinoma (IDC) cases from the Armed Forces Institute of Pathology were immunostained for pBP1 (32; Appendix). BP1 mRNA expression was found in 80% of breast tumors, compared with 11% of normal breast tissues, and, in excellent agreement, BP1 protein was detected in 81% of invasive ductal carcinomas. Surprisingly, 89% of the tumors of African American women (AAW) were BP1 positive, compared with 57% of the tumors of Caucasian women (p=0.04) (31); we have validated this association by IHC in 65 additional patients (data not shown). These results are potentially important in light of the poorer survival of AAW with breast cancer compared with Caucasian women (33). In addition, 100% of ER negative tumors were BP1 positive, compared with 73% of ER positive tumors, a significant difference (p=0.03); ER negative tumors have a poorer prognosis than ER positive tumors (31).

(2) BP1 expression correlates with the progression of breast tumors.

The frequency of BP1 positivity, distribution and intensity of BP1 expression all increased with the progression of tumor development (normal hyperplasia in situ invasive), from a few randomly distributed BP1 positive cell clusters in normal controls to the vast majority of cells in the invasive tumors showing distinct BP1 immunoreactivity (32; Appendix). The number of BP1 positive cases increased from 0% (normal) to 21% in hyperplasia, 46% in DCIS, and 81% in IDC (p<0.0001).

(3) Expression of BP1 is associated with tumor grade.

Women with grade III tumors are almost 18 times more likely to be BP1 positive than women with grade I tumors (p=0.011), and women with one or more positive lymph nodes were 3.2 times more likely to be BP1 positive than women without positive lymph nodes (p=.o43), both powerful prognostic factors.

(4) Expression of BP1 is associated with metastasis.

Forty-six cases of inflammatory breast cancer, a rare but extremely aggressive form of breast cancer, were immunostained for BP1 expression, as well as matched lymph nodes in nine metastatic cases. All 46 cases were BP1 positive, as were the nine lymph nodes, providing the first evidence that BP1 is expressed in metastasis (P.B., Y-G. Man, A. Schwartz and P. Levine, in press). Also, analysis of 105 cases of IDC demonstrated an association between BP1 positivity and positive lymph node status (p=0.03) (P.B. and E. Jewell).

(5) Overexpression of BP1 confers an aggressive phenotype on breast cancer cells.

MCF7 cell lines were engineered to overexpress BP1 RNA and protein. Four assays were used to test the aggressiveness of these cells; all indicated that high BP1 expression resulted in a more aggressive phenotype. BP1 overexpression resulted in: (i) an increased ability of cells to grow in the absence of serum; (ii) significantly larger colonies in soft agar; (iii) increased invasion through matrigel; (iii) both increased numbers of tumors and the ability of some tumors to grow in the absence of estrogen in mice, indicating estrogen independence.

(6) Overexpression of BP1 confers resistance to cell death in breast cancer cells

TNF-a is a cytokine that induces apoptosis through the death receptor pathway (34). Overexpression of BP1 increased the viability of MCF7 cells treated with TNF-a approximately two-fold (P. B. and H. Stevenson, in press). Resistance to TNF-a was due to binding of pBP1 to the regulatory region of the bcl-2 gene, a death suppressor (35), resulting in elevated expression of Bcl-2 protein. High Bcl-2 levels are associated with resistance to drug and radiation therapy (36). BP1 expression may contribute to malignancy through a reduction of apoptosis.

(7) pBP1 is secreted.

It is well documented that homeotic proteins can be secreted (37). We have strong evidence that pBP1 is secreted and thus could be detectable in serum (Preliminary Data).

### Other Analytes

Besides serum proteins/cancer markers, additional analytes of interest contemplated as within the scope of this invention include without limitation cardiac troponin, myoglobin, creatinine kinase, creatine kinase isozyme MB, albumin, myeloperoxidase, C-reactive protein, glucose, hemoglobin, hematocrit, nucleic acids and polynucleic acids, peptides and polypeptides, small molecule pharmaceuticals, industrial chemicals, bio and chemical toxins, biowarfare agents, chemical warfare agents, and the like, anything related to any of the above items, and combinations thereof.

### Experimental Data

SAW device development.

Conventional SAW devices are typically built by depositing a piezoelectric material (quartz, lithium tantalite, lithium niobate, zinc oxide) on a substrate and patterning the interdigital transducer (IDTs) on top of the piezoelectric film as depicted in FIG. 2. This sequence of fabrication does not comply with any commercially available Integrated Circuits CMOS process as it requires an extra step to deposit the piezoelectric material. It has been reported that enhanced electromechanical coupling is theoretically possible using piezoelectric films overlaying interdigital metal electrodes (39). Therefore, we have employed IDT under the piezoelectric material. By depositing the piezoelectric material on top of the IDTs as a post processing step, circuits on the same substrate are integrated with the sensors to realize smart systems.

Manufacturing Methods CMOS is short for complementary metal oxide semiconductor. Pronounced see—moss, CMOS is a widely used type of semiconductor. CMOS semiconductors use both NMOS (negative polarity) and PMOS (positive polarity) circuits. Since only one of the circuit types is on at any given time, CMOS chips require less power than chips using just one type of transistor. This makes them particularly attractive for use in battery-powered devices, such as portable computers. Personal computers also contain a small amount of battery-powered CMOS memory to hold the date, time, and system setup parameters.

SAW (surface acoustic wave) devices are widely used as electronic filters, delay lines, resonators in today's communication systems. Although telecommunication industry is the largest user of these devices, SAW based sensors have many attractive features to be explored for emerging technologies in automotive (torque, pressure), medical (biosensor) and commercial (vapor, gas, humidity) applications.

Surface acoustic waves (both Rayleigh and pseudo-SAW) are generated at the free surface of a piezoelectric material. An application of a varying voltage to the metal IDT (interdigital transducer) generates the acoustic wave on the input side. In the basic configuration there is an input IDT and an output IDT. The acoustic wave generated by the input IDT travels through the region called the delay line and reaches the output IDT where the mechanical displacements due to the acoustic waves create a voltage difference between the output IDT fingers. One of the most widely used and interesting sensing mechanism that acoustic wave sensors employ is mass loading. Prominent applications are in film thickness monitoring, gas, liquid phase chemical sensing and biosensing. The delay lines of SAW devices are coated with some bio/chemical coating which selectively reacts with the entity under analysis. This interaction produces a shift in the resonant frequency of the SAW device. By measuring this shift in frequency domain, a detailed analysis of the entity being sensed can be completed.

Using a combination of IC compatible technologies, such as Si micromachining, thin film deposition, bio/chemical layer growth, integrated electronics, smart structures and systems can be realized. Considering the advantages that CMOS technology provides along with the ever-developing CMOS compatible MEMS processes, the SAW technology performance can be improved significantly. Therefore, an array of SAW delay lines were designed and fabricated through a regular CMOS process sequence, characterized and post-processed using widely used MEMS techniques.

### Design

The mass sensitivities of different acoustic devices are related to structure geometry, resonant center frequency, and electromechanical coupling. The most important specification for SAW device design is the center frequency, which is determined by the period of the IDT fingers and the acoustic velocity of the piezoelectric material. The governing equation that determines the operation frequency

vSAW=λ×fc (1)

λ: the wavelength at fc, determined by the periodicity of the IDT.
fc: the center frequency of the device
vSAW: the velocity of the SAW

For the case of devices that were tested

λ=p=finger width×4=2.4 μm×4=9.6 μm (2)

Based on tabulated data and calculations for the corresponding design dimensions vSAW for ZnO is 3820 m/s. This translates into a center frequency of fc=3820 m/s/9.6 μm=397.916 MHz (3)

### Saw Fabrication Sequence and Results

As stated, the conventional SAW devices are typically built by depositing a piezoelectric material (quartz, lithium tantalite, lithium niobate, zinc oxide) on a substrate and patterning the IDTs on top of the piezoelectric film. Since the conventional sequence of fabrication requires an extra step to deposit the piezoelectric material, we employed the piezoelectric films overlaying the interdigital metal electrodes. Thus, the placement of IDT under the piezoelectric material is employed in certain preferred embodiments for this work. By depositing the piezoelectric material on top of the IDTs as a post processing step, the CMOS process is not disturbed.

For device stability as a function of temperature it is essential that the temperature coefficient of delay (TCD) be as small as possible. The slope of the TCD as a function of temperature for SAW substrates is mainly negative, so that an increase in temperature will cause a downward shift in IDT center frequency and vice-versa. Due to this strong temperature dependence of the device performance and for precise control of the temperature to study the effects of temperature on the mass sensitive layer and the analyte of interest, a novel heater design is developed. For the heater elements n-well was picked as the resistive layer. The n-well layer has a TCR of 0.5-0.75%/K, which is the highest among various CMOS process layers. Moreover, n-well provides an embedded heater structure that can directly control the temperature of the substrate and the mass sensitive area without causing any disturbance on the SAW delay line path or the IDT finger design as in the case of other candidate layers (e.g. polysilicon, metal) for resistive heating.

The major distortions in the transfer characteristics of SAW devices occur due to interference of the reflected waves and the triple transit effect. In Rayleigh wave devices, acoustic absorbers were shown to be effective against the reflections. They consist of soft materials located on the surface, at the edges of the device. In order to investigate the performance of the SiO₂ in reflecting or attenuating acoustic waves, an absorber structure was designed from stacking CMOS layers of metal1, metal2 and polysilicon. By stacking these dummy strips, a surface higher than the IDT level was created which is investigated for attenuating or reflecting the acoustic waves.

### A. Step 1:Removal of Oxides

After the chips are fabricated, they are covered with the overglass protection and the dielectrics (SiO₂) over and underneath the metal and poly layers. The first step in the fabrication is RIE (Reactive Ion Etch). This step has been developed, examined, and characterized previously. Note that AMI 1.5 μm process contains only two metal layers, one of which is already being used as IDTs in this run and the second metal layer will be used in the absorber structures for two main purposes. 1) To increase the height of the absorber compared to the height of the IDTs 2) To act as a mask layer that protects the oxide, which is built up under this layer. The RIE etches the dielectric that is not covered with aluminum, including field oxide, overglass, and intermetal dielectrics. The dies that were fabricated through MOSIS were RIE etched through MEMS-Exchange. The etching was carried out in a Plasma Therm 72 RIE equipment under 40 mTorr pressure.

The etch rate is 250 Å/min. The depth of the total oxide removed by the etch process is 1.5 μm. After the RIE step is completed, three major areas are defined on the surface. The absorbers, the IDTS (both of which are expected to retain their dielectric layers underneath) and exposed Si, which lay between the IDTs and the area that will define the delay line.

### B. Step 2:Sputtering of ZnO

ZnO finds wide applications in SAW devices due to its strong piezoelectric effect among non ferroelectric materials. A variety of deposition techniques were used for the growth of ZnO on various substrates. Among them sputtering is considered to be the most favorable one as it is possible to obtain well oriented and uniform ZnO films. Therefore, RF magnetron sputtering was one preferred choice of deposition.

By maskless sputtering from the front, the ZnO covers the entire surface including the IDT fingers and the exposed Si. This inevitably creates bulks of ZnO layers between the IDT fingers. Sputter deposition of zinc oxide shows superior properties over other deposition methods, but the quality of the sputtered film and the growth constitutes a major interest in SAW related applications. The ZnO was deposited on the previously etched dies through MEMS-Exchange. The targeted thickness was 3.0 μm and the measured film thickness variation was (+/−%) 13.1. Argon and oxygen were used with a set-up time of 180 min in an MRC Sputter at 200° C. The surface morphology, roughness and the crystal orientation of the sputtered ZnO are subject of interest. Therefore, a thorough characterization of the sputtered films is required. The ZnO covered samples are analyzed for the crystal orientation in Scintag XRD 1000. For comparative analysis, 2θi scans of a dummy die (Si—ZnO) and a patterned die (Si—SiO2-Al—ZnO) were carried out. As it can be calculated, the ZnO shows its peak at 2θi=34.721, which agrees with the tabulated data in the literature for (002) 2θi=34.421. It also has a peak at 2θi=73, which corresponds to its listed peak at 2θi=72.560 for (004). In order to obtain information regarding the surface roughness and grain size JOEL High Vacuum Integrated STM/AFM/JSPM-5200 was utilized. The multilayered (Si—SiO2-Al—ZnO) dies were characterized.

### C. Step 3: Patterning of the Pad Frame/ZnO Etching

After the ZnO deposition the entire die is covered with the sputtered ZnO. In order to access the pads for bonding a last step of lithography and etching are required. The entire die area is 2.2 mm×2.2 mm and the pads are 100 μm×100 μm. A simple shadow mask idea was employed for the etching step. The mask was constructed by using a square Si piece of size 2×2 mm. The major problem encountered during the patterning was the photoresist build up on the edges of the die. Thickness measurements showed a 2-4 μm phototresist buildup on the pad frame region when the thickness on the areas closer to the center of dies were measured to be 1 μm. In order to completely remove the photoresist build up covering the pad frame, a two step process was carried out. In the first step, Shipley 1818 2:1 thinner was spun for 40 sec at 5000 rpm and the samples were exposed for 20 sec. Then a 2 min development in 5:1 Developer was applied. In the second step, the same exposure time and development time was used to remove the excessive photoresist.

In general, ZnO is attacked by all common acids and bases. In order to achieve smooth etch profiles and minimize the undercutting, the etching process should be slowed. Therefore, a very dilute solution of two acids was used. The most common acids that are listed in the literature are acetic, hydrocloric and phosphoric acids. A solution of H₃PO₄: CH₃COOH: D₁-H₂O with 1:1:150 was used for the etching.

2.6 μm thick ZnO was etched completely in 4 min, which translates into 648 Å/min etch rate. Once the final step of post processing was completed, the dies were bonded on a DIP-40 package for electrical testing. The major subjects of interest for performance analysis of SAW devices are the transmission coefficient and the reflection coefficient versus frequency. HP 8712ET, 300 kHz-1300 MHz, RF Network Analyzer was used for this purpose. A through-cable calibration was carried out in order to measure and compensate for the losses and errors due to the connector and cable irregularities. Thus, a test chip that contains an array of SAW delay lines were designed and fabricated in AMI 1.5 μm 2 metal, 2 poly process. A unique, three step, maskless post processing sequence was developed. Complete characterization of the two etching steps and piezoelectric film was carried out. The transfer characteristics show a maximum transmission and minimum reflection at 392.5 MHz with an insertion loss of −4.83 dB. The 3 dB bandwidth was measured to be 19.25 MHz which agrees closely with the calculated value of 14.575 MHz. The results demonstrated the success of one preferred design and the ability to fabricate SAW based sensors with comparable performance to conventional devices by using any commercially available CMOS technology.

### CMOS Process Sequence

CMOS fabrication technology is well established and requires that both n-channel (nMOS) and p-channel (pMOS) transistors be built on the same chip substrate. To accommodate both nMOS and pMOS devices, special regions must be created in which the semiconductor type is opposite to the substrate type. These regions are called wells or tubs. A p-well is created in an n-type substrate or, alternatively, an n-well is created in a p-type substrate. In the simple n-well CMOS fabrication technology presented, the nMOS transistor is created in the p-type substrate, and the pMOS transistor is created in the n-well, which is built-in into the p-type substrate. In the twin-tub CMOS technology, additional tubs of the same type as the substrate can also be created for device optimization.

The simplified process sequence for the fabrication of CMOS integrated circuits on a p-type silicon substrate starts with the creation of the n-well regions for pMOS transistors, by impurity implantation into the substrate. Then, a thick oxide is grown in the regions surrounding the nMOS and pMOS active regions. The thin gate oxide is subsequently grown on the surface through thermal oxidation. These steps are followed by the creation of n+ and p+ regions (source, drain and channel-stop implants) and by final metallization (creation of metal interconnects).

An integrated circuit (IC) is a circuit comprised of elements such as transistors, resistors and capacitors fabricated in a single piece of semiconducting material, usually silicon or gallium arsenide. As used herein, “integrated circuit” not only refers to the common definition but also to highly integrated structures including, for example:

1) multichip modules where several IC's and other circuit elements including molecular target probes may be combined compactly on a polymer, quartz, glass, sliver, ceramic or other substrates. In some cases, one IC may be the substrate with other components, such as photodiodes or LEDs mounted on it;

2) Hybrid microcircuits where one or more IC's and other circuit elements are mounted on or several substrate(s); and,

3) Other compact electromechanical arrangements of a circuit comprising primarily one but possibly more IC's and other electronic components and microelectromechanised systems (MEMs).

### Surface Acoustic Wave Resonators

Surface acoustic wave (SAW) resonators have commonly been used as the local oscillator (LO) which is one of the basic building blocks of the RF receiver heterodyne architecture and is currently the major stumbling block for complete integration of RF receivers. Due to the structure of the SAW devices that require fabrication of metallized interdigital transducers on piezoelectric crystals, the SAW resonators are difficult to implement in CMOS and are usually realized as discrete off-chip components. Efforts to integrate surface acoustic wave filters on silicon substrates have been previously implemented by Visser, Vellekoop and Zeijl while the monolithic integration of SAW devices on GaAs has been implemented by Baca of Sandia National Labs.

The operating frequency of a surface acoustic wave resonator is determined by the periodic distance of its interdigitated transducer (IDT) fingers, λ, which are placed on piezoelectric material. Unlike bulk-wave crystal resonators, whose frequency of operation is determined by the thickness of the piezoelectric material, surface acoustic wave resonators have a more area efficient structure since its frequency of operation is determined by the spacing between the IDTs and thus is only limited by the resolution of the fabrication process. The utilization of surface waves, instead of bulk-waves, makes the process of integration with the present integrated circuit fabrication techniques more feasible since it eliminates the necessity of having a thick piezoelectric layer sandwiched between two metal layers.

In the present invention, implementation of surface acoustic wave resonators using the standard CMOS fabrication technology with additional post-processing techniques is provided. The fact that the fabrication process of this SAW resonator is highly compatible with current integrated circuit processing techniques makes it extremely desirable since it is not only cost-effective, but also allows the resonator features to be less prone to manufacturing defects due to the maturity of CMOS technology. In this design, the SAW resonator utilized the minimum feature sizes possible in the available 0.6 micron AMI CMOS process to realize a 1.15 GHz surface acoustic wave resonator.

The fabricated CMOS resonators were measured, and modeled as a two-port electrical network through analog circuit synthesis. This model was used as a basis for simulation in Cadence to design a Pierce oscillator. The Pierce amplifier was designed on a separate CMOS chip. When connected together, the Pierce amplifier and SAW resonator realize an oscillator capable of synthesizing a frequency of 1.15 GHz. By having both the resonator and the oscillator integrated on the same CMOS chip, a fully monolithic frequency synthesizer is realized.

CMOS Surface Acoustic Wave Resonators: Design and Equivalent Circuit Model

### A. SAW Resonator Design

The SAW resonator design consists of input or output IDTs flanked by a bank of reflectors on both sides. The key design parameter of a one port resonator is λ, or the periodic distance between two interdigitated fingers, which determines the frequency of the resonator, using v=fλ, where v is the velocity of the acoustic wave in the piezoelectric material and f is the resonant frequency. In this design run, λ was chosen to be four times the minimum metal2 size of this technology or 3.6 micron. The modeling equations for SAW resonators, which involve rigorous computations of transmission matrices, will not be discussed in detail in this paper but the basic methodology of designing the SAW resonators and its accompanying oscillator circuit is outlined here.

The main design parameter that needs to be chosen is M or the number of reflectors. An array of reflectors is required since each electrode only produces reflectivity, r of approximately 0.08. The equation for reflectivity was derived based on the theory of reflection in transmission lines, and is given in (1A), where η is the metallization ratio of the IDT, Z is the characteristic impedance of the region under the reflector, ΔZ is the difference of characteristic impedance between the free region and the region under the reflector, and finally, fᵣ is the resonance frequency.

$$\begin{matrix}r = j \frac{Δ Z}{Z} sin ( η π \frac{f}{fr} ) & ( 1 ) ( 1 A ) \\ Γ ∼ tanh ( M ) & ( 2 A ) \\ L = 1 / ( 4 \vert r \vert ) & ( 3 A )\end{matrix}$$

The array reflectivity, Γ can also be approximated as (2A) and the number of reflectors was chosen to optimize area while maintaining array reflectivity to be close to 1. The effective center of reflection, or the line where the surface waves are assumed to be totally reflected is denoted by L is expressed as (3A) where r is the reflectivity of each electrode. MATLAB simulations of (1A), (2A) and (3A) using ZnO thin film material properties were performed to obtain the optimum number of reflectors which was found to be 125 for this design. The total effective length of a round trip in the cavity must be an integer number of λ. Other important design parameters are the distance between the reflectors and the input transducers Lg, the number of IDTs or Nₜ and the width of the transducers or W. Thirty-two different designs of SAW resonators were first fabricated on piezoelectric substrates such as lithium niobium substrates to evaluate the performance of the fabricated designs. Four resonators with the best performance characteristics were then chosen and scaled to fit the 0.6 um CMOS in this design run.

### B. Equivalent Circuit Model

The equivalent circuit model of a two-port SAW resonator was developed using analog circuit synthesis based on the S21 frequency measurements made on the fabricated CMOS resonators. The SAW resonator has transmission characteristics similar to a series LCR circuit, where the series resonant frequency is generated by both Lₓ and Cₓ. The initial values for the LCR circuit were calculated using the resonator design equations based on the reflectivity of each metal strip and number of reflectors. This basic LCR circuit was then modified based on curve fitting, where the simulated values were compared with the experimental measurements. Several circuit topologies were implemented an least squares method was used to choose the circuit, which produced the best fit. The final circuit topology that produced the best fit is used and may consist of Rx, Cx and Lx which are motional resistance, capacitance and inductance connected in parallel with Lp and Rp. The input and output ports are connected with parasitic capacitances Cp to ground. The circuit's S21 scattering parameters analysis simulations were performed using Cadence SpectreRF where the input and output ports were terminated with 50 Ohms. Comparison between the measured and simulated equivalent circuit model is possible. The best fitting curve was found to have the values of Lx=1 uH, Rx=120 Ohms, Cx=19 fF, Lp=132.8 nH, and Cp=10 fF.

### Fabrication

One important highlight of this SAW resonator design is that the SAW resonator IDT's are implemented using standard CMOS technology. This allows the resonator features to be realized in the minimum sizes available in the AMI 0.6 micron CMOS process, making them not only less susceptible to manufacturing defects but also less expensive compared to when fabricated as micromachined MEMS structures. The resonator features were implemented as 0.9 micron wide metal2 lines which are able to realize ultra-high frequency, 1.15 GHz resonators.

The SAW CMOS post fabrication sequence is similar to the CMOS SAW delay line, with several important distinctions, namely the presence of the aluminum ground metal1 shield which was placed to eliminate electromagnetic feed-through. The other major difference is the presence of the oscillator circuit which is also placed on the same die and protected by a grounded metal layer.

Once the standard CMOS fabrication sequence is completed, the first post-processing step involves releasing the SAW IDTs from the insulating SiO₂ layer using reactive ion etch (RIE). The next step is ZnO deposition, which was sputtered on the resonators. The final post-processing step involves wet-etching the ZnO to uncover the resonators' pads.

As examples, two separate CMOS chips had been designed. The first chip (Chip A) consists of four CMOS SAW resonators was fabricated and characterized prior to the design of the second chip. Chip B which consists of three resonators and the Pierce oscillator circuit. Chip A was designed to characterize the ultra high frequency resonator of frequencies in the range of 900 MHz to 1.2 GHz. Chip B was implemented as a proof of concept that the post-processing techniques of the SAW resonator do not adversely affect the amplifier circuit.

### Experimental Results and Discussion

Once the post-processing steps were completed, the die was bonded and packaged in a DIP40 chip package to facilitate measurements. The bonded device was measured using a HP8712 network analyzer. The S21 measurements made on the 0.6 micron CMOS SAW resonators produced the parallel resonance frequency, fp of 1.084 GHz and the series resonance frequency, fs of 1.156 GHz. Based on the measurements, the parallel Q was calculated to be 90.33 and series Q was calculated to be 36.125. The measured Q was not as high as expected since the die was overetched and some parts of the resonator was not completely covered. The acoustic wave velocity of ZnO calculated from the measured curve was found to be 4140 m/s.

### Oscillator Design and Simulation

To operate as a local oscillator, the SAW resonator is connected to a Pierce oscillator at the gate and drain of M1. This three-point oscillator topology was previously implemented using an FBAR oscillator by Otis, and was chosen firstly since it utilizes the current source, M2 to provide the necessary biasing current to M1, as the SAW resonator does not pass any DC current. Secondly, it also provides excellent phase noise characteristics. Mfb, operates as a resistor which provides bias to the gate of M1.

A_{CL}=gₘRₚ(Cₚ₁/Cₚ₂) (4A)

To ensure oscillation, the loop gain, A_{CL} has to be greater than 1 and the phase shift should be equal to 0. Based on (4A), the size of M1 is adjusted to provide the necessary transconductance to counteract Rₚ, where Rₚ is the parallel reflected motional resistance at the gate of M1. For this design Cp1=Cp2 which also maximizes the open loop gain. The transistors were sized as follows, M1=4.8 m/1.6 u, Mₜₕ=4 u/872 u, M2=9.6 m/1.2 u. The phase noise performance and steady-state analysis of the oscillator circuit was simulated using Cadence SpectreRF to obtain its fundamental beat frequency of 1.154 GHz.

Accordingly, the design, implementation and measurements of a CMOS SAW resonator is provided. The SAW resonators were fabricated using standard AMI 0.6 micron CMOS technology with some additional post-processing steps. Upon completion, the SAW resonators were characterized and found to have a resonant frequency of 1.15 GHz and its equivalent circuit model was developed based on these measurements. This model was used to design and simulate a Pierce 1 GHz oscillator.

### Biological Sensors

Surface acoustic wave devices as biosensors are also well suited for the detection of biological agents. Positioning a receptor between IDT fingers to induce a phase shift or within IDT fingers to induce a frequency shift allows for electronic detection of bioagents. These devices have the dual advantages of high sensitivity, down to picograms/cm2, and high specificity, conferred by biological receptors such as antibodies, peptides, and nucleic acids. In the present invention, detection of proteins associated with breast cancer are contemplated with these types of sensors. Handheld biodetection systems incorporating these microsensors are contemplated as within the scope of the invention.

The surface acoustic wave biosensor arrangements may be used for real time sensing and for quantifying the levels of protein.

Many monoclonal antibodies with high affinity and specificity for proteins associated with breast cancer, e.g. mammoglobin, BP1, among others, are available from commercial sources as well as the American Type Culture Collection (ATCC). Any known coupling chemistry may be used for binding the monoclonal antibody to the sensor chip surface.

It may also important to optimize the chemical linking of the antibody, as well as the loading density. Independent fluorescence assays of the antibody density on the chip may be done using fluorescence labeled anti-IgG. The chip may be incubated in phosphate buffered saline with fluorescein labeled anti-IgG, and may then be washed with a buffer solution of increasing ionic strength to dislodge unbound antibody. The chip may then be scanned using a Perkin Elmer LS50B fluorescence spectrophotometer, and the bound antibody density may be calculated using FL-Winlab software, which may also calculate various parameters (such as, for example, the statistical variability observed in the surface loading between regions on the chip sample surface). This technique may be used to determine which of the coupling chemistries yields the best loading of antibodies.

There are a number of U.S. patents which describe integration of a biosensor chip into a biosensor device, including 6,937,052, 6,743,581, 6,657,269, and 6,448,064, all incorporated herein in their entirety as necessary to enable the subject matter of the invention. Other U.S. patents describe the use for detection of a chemical, including 6,627,154, and 6,495,892, incorporated herein in their entirety as necessary to enable the subject matter of the invention.

SAW devices are extremely sensitive to tiny mass changes, detecting 100 pg/cm²—less than 1% of a monolayer of carbon atoms. When coated with a chemically selective thin film, the SAW device is rendered sensitive to analytes that interact with the film.

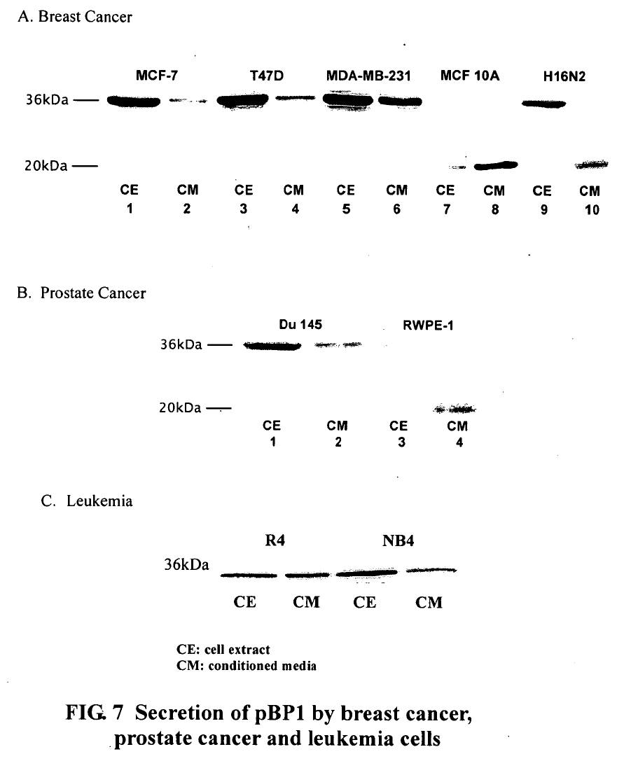

FIGS. 3, 4 and 7 directed to biosensor technology show that serum testing for cancer may be provided herein and is considered within the scope of the present invention. FIG. 7 shows that the invention is not limited to any one particular target analyte and that data for multiple cancers illustrate this to the degree that a person of ordinary interest in this field would understand that the invention is not limited to a single target biomolecule or marker or a single type of cancer.

The references recited herein are incorporated herein in their entirety, particularly as they relate to teaching the level of ordinary skill in this art and for any disclosure necessary for the commoner understanding of the subject matter of the claimed invention. It will be clear to a person of ordinary skill in the art that the above embodiments may be altered or that insubstantial changes may be made without departing from the scope of the invention. Accordingly, the scope of the invention is determined by the scope of the following claims and their equitable Equivalents.

### REFERENCES

Duffy, M. J. (2006) Serum Tumor Markers in Breast Cancer: Are They of Clinical Value? Clinical Chemistry 52: 345-351.
Ross, J. S., Linette, G. P., Stec, J., Clark, E., Ayers, M., Leschly, N., Symmans, W. F., Hortobagyi, G. N., and Puszati, L. (2003) Breast Cancer Biomarkers and Molecular Medicine. Expert. Rev. Mol. Diagn. 3: 573-585.
Serengi, E., Coli, A., and Mazzucca, N. (2004) Circulating Tumour Markers in Breast Cancer. European Journal of Nuclear Medicine and Molecular Imaging 31: S15-S22.
Cianfrocca M., and Goldstein, L. J. (2004) Prognostic and predictive factors in early-stage breast cancer. Oncologist 9:606-16.
Bull, S. B., Ozcelik, H., Pinnaduwage, D., Blackstein, M. E., Sutherland, D. A., Pritchard, K. I., Tzontcheva, A. T., D. R., and Andruis I. L. (2004) The combination of p53 mutation and neu/erbB-2 amplification is associated with poor survival in node-negative breast cancer. Clin Oncol. 22:86-96.
Bernstein, J. L., Godbold, J. H., Raptis, G., Watson, M. A., Levinson, B., Aaronson, S. A. and Fleming, T. P. (2005) Identification of mammaglobin as a novel serum marker for breast cancer. Clin. Cancer Res. 11: 6528-6535.
Mascini, M. “Coupling proteins or oligonucleotides with QCM-Optimization of strategies” Universita degli Studi di Firenze. HREF=“http://www.chim.unifi.it/ana”§ MACROBUTTON HtmlResAnchor http://www.chim.unifi.it/ana
Tigli, O. and Zaghloul, M. E. (2007) A Novel SAW Sensor in CMOS: Design, Modeling, and Fabrication”, IEEE Sensors Journal 7 (2): 219-227.
Kingsmore, S. F. (2006) Multiplexed protein measurement: technologies and applications of protein and antibody arrays. Nature Rev. Drug Discovery 5: 310-320.
Saaristo, A., T. Karpanen, et al. (2000) Mechanisms of angiogenesis and their use in the inhibition of tumor growth and metastasis. Oncogene 19: 6122-6129.
Salven, P., V. Perhoniemi, et al. (1999) Serum VEGF levels in women with a benign breast tumor or breast cancer. Breast Cancer Res Treat 53: 161-166.
Heer, K., H. Kumar, et al. (2001) Serum vascular endothelial growth factor in breast cancer: its relation with cancer type and estrogen receptor status. Clin Cancer Res 7: 3491-3494.
Fu, S., Schwartz, A., Stevenson, H., Pinzone, J. J., Davenport, G. J., Orenstein, J. M., Gutierrez, P., Simmens, S., Abraham, J., Poola, I., Stephan, D. A. and Berg, P. E. (2003) Correlation of expression of BP1, a homeobox gene, with estrogen receptor status in breast cancer. Br. Ca. Res. 5:82-87.
Man, Y-g., Fu, S. W, Schwartz, A., Pinzone, J. J., Simmens, S. J. and Berg, P. E. (2005) Expression of BP1, a novel homeobox gene, correlates with breast cancer progression and invasion. Br. Ca. Res. & Treat. 90: 241-247.
Stevenson, H. S., Fu, S., Pinzone, J. J., Campbell, C. L., Simmens, S. J. and Berg, P. E. (2007) BP1 transcriptionally activates bcl-2 and inhibits TNFα-induced cell death in MCF-7 breast cancer cells. Br. Ca. Res. 9: R60.
Dickson, R. B. and Lippman, M. E. (1995) Growth factors in breast cancer. Endocrine Rev. 16: 559-589.
Prochiantz, A. and Joliot, A. (2003) Can transcription factors function as cell-cell signaling molecules? Nature Rev. Mol. Cell. Biol. 4: 814-819.
Haab, B. B. (2005) Antibody arrays in cancer research. Molec. Cell. Proteomics 4.4: 377-383.
Martin, V. S., Sullivan, B. A., Walker, K., Hawk, H., Sullivan, B. P. and Joe, L. J. (2006) Surface Plasmon resonance investigations of human epidermal growth factor receptor 2. Appl. Spectroscopy 60: 994-1003.
Span, P. N., et al (2004) Mammaglobin is associated with low-grade, steroid receptor-positive breast tumors from postmenopausal patients, and has independent prognostic value for relapse-free survival time. J. Clin. Oncol. 22: 691-698.
Cheung, K. L., Graves, C. R. L. and Robertson, J. F. R. (2000) Tumour marker measurements in the diagnosis and monitoring of breast cancer. Ca. Treat. Rev. 26: 91-102.

We claim:

1. A biosensor device, comprising: a CMOS integrated circuit having a SAW device as an on-chip component and configured for an analyte detection assay, wherein the SAW device has an Si substrate having a piezoelectric film thereon, said film supporting an input IDT and an output IDT in cooperative association, and having a biochemical coating there-between for detecting at least one analyte; wherein the biochemical coating acts as an analyte capture binding surface, and wherein upon binding of said analyte to said biochemical coating a surface acoustic wave signal is generated that is indicative of the presence of the analyte.

2. The biosensor device of claim 1, further comprising wherein the device is configured for parallel multi-analyte, multi-concentration detection assays.

3. The biosensor device of claim 2, wherein the configuration comprises wherein the piezoelectric film comprises a micromachined matrix of addressable microlocations for analyte capture.

4. The biosensor device of claim 1 or 2, further comprising wherein the biosensor has a plurality of detection channels, said detection channels having a plurality of biochemical coatings specific for detection of a plurality of proteins, and which provides for a multi-protein multi-concentration detection array capable of parallel simultaneous read-out.

5. The biosensor device of claim 1, wherein the biochemical coating is for detecting at least one serum tumor marker protein for cancer.

6. The biosensor device of claim 1, wherein the piezoelectric film is deposited on top of the IDT.

7. The biosensor device of claim 1 or 2, wherein the device is configured to provide an electronic readout of the concentration of serum tumor marker as determined from the strength of the SAW signal generated.

8. A method of detecting biomolecules, comprising: contacting the analyte capture binding surface of the biosensor device of claim 1 with a sample of interest.

9. The method of claim 5, wherein the sample is a serum protein cancer marker.

10. The biosensor of claim 1, further comprising a CMOS manufactured embedded heater under the SAW sensor, wherein the heater is controlled by applying outside voltage, and wherein control of the sensor temperature to the desired value is provided to keep the sensor operating point stable over time.

11. The biosensor of claim 1, further comprising wherein the analyte is a marker protein selected from the group consisting of mammoglobin, BP1, CEA, CA15-3, TPA, TPS, HER-2, and combinations thereof.

12. The invention according to claim 1 or 2, further comprising wherein the biosensor device provides an electronic readout and is implemented in a single unit.

13. A method of detecting a serum tumor marker for breast cancer, comprising:
a) providing a biosensor of claim 1;
b) contacting the biochemical coating of the biosensor with a sample; and
b) detecting the SAW signal,
wherein upon binding of a serum tumor marker to said biochemical coating then a surface acoustic wave signal is generated that is indicative of the presence of the serum tumor marker protein, and wherein upon the absence of binding of the serum tumor marker, then no signal is generated and indicates the absence of said serum tumor marker protein.

14. A method of detecting multiple proteins in samples having multiple concentrations in a multiplex biosensor, comprising:
a) providing the multiplex biosensor of claim 4;
b) contacting each of the plurality of detection channels of the biosensor with sample; and
b) detecting the SAW signal,
wherein upon binding of a specific serum tumor marker to said biochemical coating then a specific surface acoustic wave signal is generated that is indicative of the presence of the specific serum tumor marker protein, and wherein upon the absence of binding of the specific serum tumor marker, then no signal is generated and indicates the absence of said specific serum tumor marker protein, and wherein the multiplex biosensor provides for a multi-protein detection array capable of parallel simultaneous read-out.

15. The method of claim 8, 13 or 14, further comprising the step of determining the concentration of the analyte or serum tumor marker from the strength of the SAW signal generated.
